(19) United States
(12) Patent Application Publication
(10) Pub. No.: US 2003/0176356 A1
(43) Pub. Date: Sep. 18, 2003

(54) Endothelin antagonists and endothelin-converting enzyme inhibitors for the treatment of glaucoma

(75) Inventors: Thomas Yorio, Burleson, TX (US); Ganesh Prasanna, Fort Worth, TX (US); Adnan Dibas, Fort Worth, TX (US); Martha E. Stokely, Fort Worth, TX (US)

Correspondence Address: JACKSON WALKER LLP, 2435 NORTH CENTRAL EXPRESSWAY, SUITE 600, RICHARDSON, TX, 75080, US

(73) Assignee: University of North Texas Health Science Center, Fort Worth, TX

(21) Appl. No.: 10/131,787

(22) Filed: Apr. 24, 2002

Related U.S. Application Data
(60) Provisional application No. 60/285,960, filed on Apr. 24, 2001.

(51) Int. Cl. A61K 38/08; A61K 31/519

(52) U.S. Cl. 514/17; 514/259.1

(57) ABSTRACT

A pharmaceutical composition, containing a therapeutically effective amount of an endothelin (“ET”) antagonists and/or an endothelin-converting enzyme (“ECE”) inhibitors and a pharmaceutically acceptable carrier. The pharmaceutical composition is useful for treating normal-tension and primary open-angle glaucoma and prevents optic nerve damage and retinal ganglion cell death associated with these ocular diseases.

This application claims priority to a provisional patent application 60 285 960, filed on Apr. 24, 2001.

The government may own certain rights in the present invention pursuant to grant number EY11979 from NIH/NEI and 009768-018 from Advanced Research Program-Texas.

The present invention related generally to the fields of glaucoma therapy. More particularly, it concerns the ability of certain agents to block (e.g. antagonists) or prevent the synthesis (e.g. endothelin-converting enzyme inhibitors) of endothelin (ET), a small but very potent protein. These agents would prevent endothelins' ability to promote optic nerve damage that leads to optic nerve neuropathy, which is characteristic of glaucoma.

### BACKGROUND

There are many common types of ocular diseases or damages known to profoundly affect human vision (e.g. glaucoma, ischemic retinopathies, optic neuropathies, commotio retinae, macular degeneration, retinitis pigmentosa, retinal detachment, retinal tears or holes, diabetic retinopathy, iatrogenic retinopathy, and others). However, each of the general terms for such diseases or damages may only represent a broad spectrum of conditions. For example, glaucoma is the term used for a diverse group of eye diseases, all of which result in blindness due to the progressive damage to the optic nerve. Optic nerve damage produces certain characteristic defects in the individual's peripheral vision, or visual field. Glaucoma is usually, but not always, accompanied by elevated intraocular pressure (“IOP”) of the fluid called aqueous humor. Typically in glaucoma, there is a build-up of resistance during the clearance of aqueous humor from the eye, which causes the IOP to become elevated. There are three basic types of glaucoma: primary, secondary and congenital. Primary glaucoma is the most common type and can be divided into open-angle and closed-angle glaucoma. Primary open angle glaucoma (“POAG”) is the most frequent type observed in the United States. POAG is usually detected in its early stages during routine eye examinations. Primary closed angle glaucoma, also called acute glaucoma, usually has a sudden onset and is characterized by eye pain and blurred vision. Secondary glaucoma occurs as a complication of a variety of other conditions, such as injury, inflammation, vascular disease and diabetes. Congenital glaucoma is due to a developmental defect in the eye's drainage mechanism.

The three basic types of glaucoma described above are a leading cause of blindness worldwide (66 million people). At least six known glaucoma genes have been identified, which provides a genetic linkage to some of these common forms of glaucoma. Recently, a tissue-specific stress response factor, called endothelial leukocyte adhesion-1 (“ELAM-1”) molecule, was shown to be markedly increased in the trabecular meshwork of glaucomatous eyes of diverse etiologies. ELAM-1 is putatively involved in protecting cells against oxidative stress and is a characteristic early marker for atherosclerotic plaques in the vasculature. A similar finding in glaucomatous trabecular meshwork cells indicates that a common pathophysiological mechanism may exist between vascular and glaucomatous diseases. Although changes in the outflow pathway cause increased intraocular pressure (“IOP”) in primary open-angle glaucoma (“POAG”), the actual mechanism responsible optic nerve damage in POAG is unclear. Although not wanting to be bound by theory, mechanisms that may contribute to the optic nerve damage in glaucoma are:

(a) the mechanical effect of IOP elevation;

(b) ischemia or vascular dysregulation;

(c) distinct cellular responses to glaucoma stimuli of ganglion cells, nerve fibers, and other cell types (amacrine, astrocytes, and lamina cribrosa); and

(d) abnormal effects of endogenous substances such as glucocorticoids, glutamate, nitric oxide, and endothelin.

Endothelins (“ET”) are potent vasoactive peptides that are implicated in the development and progression of certain forms of glaucoma (e.g. primary open-angle and normal tension glaucomas), which results in the apoptotic death of retinal ganglion cells and progressive cell death leads to blindness. It has recently been shown that patients with normotensive glaucoma have elevated plasma ET-1 concentrations and patients with primary open angle glaucoma (“POAG”) have elevated ET-1 concentrations in aqueous humor (Noske et al., 1997; Sugiyama et al., 1995). Chronic perineural administration of endothelin to the area adjacent to the optic nerve causes the vasoconstriction of the anterior optic nerve vasculature, leading to optic nerve ischemia (Cioffi et al., 1995). In addition, it has been reported that low doses of endothelin (“ET”), administered intravitreally (i.e. behind the lens into the vitreous cavity), produces similar ocular nerve damage and optic nerve cupping as seen in glaucoma (Orgul et al., 1996). Additionally, a single intravitreal injection of ET results in a significant augmentation of protein and membrane-bound organelle transport along the axon of the rat optic nerve (Stokely et al., 2002). Defective axonal transport is another characteristic feature of glaucoma, which leads to cell death of retinal ganglion cells.

Endothelin-induced ischemia in the retina promotes retinal ganglion cell death along two pathways: 1) ET causes the production of glutamate, an excitatory amino acid, which in excessive amounts can damage retinal ganglion cells by initiating calcium-dependent apoptotic mechanisms, and 2) ET promotes the production of nitric oxide (“NO”) that combines with oxygen free radicals to form reactive peroxynitrites, which initiate retinal ganglion cell death. There is considerable evidence to support the destructive roles of glutamate and NO in retinal ganglion cell death during glaucoma (Dreyer et al., 1996; Nathanson and McKee, 1995; Neufeld et al., 1997; Neufeld, 1999). We have shown that ET increases both NO production and nitric oxide synthase-2 activity in ocular cells (See FIGS. 4 and 5). Endothelin production occurs within many ocular cells including those of the retina (retinal pigment epithelium and astroglial cells) (Dreyer et al., 1996; MacCumber et al., 1991). As a general theme in most cells, biologically active endothelin is produced by the conversion of a precursor big endothelin (“Big ET”), which is relatively inactive, by the action of an endothelin-converting enzyme (ECE-1a, -1b, -1c and ECE-2). The current invention will utilize specific ECE inhibitors to decrease ET production in the eye and regulate ET levels or utilize ET receptor antagonists to prevent ET actions and ultimately mitigate the ill effects of ET-induced glaucomatous optic neuropathy.

The present-day drugs and surgery to treat glaucoma are limited by their actions as they mitigate only the major symptom of the disease, which is elevated intraocular pressure due to blockage of the outflow pathway as seen in primary open angle glaucoma. These drugs do not target the site of damage i.e. prevent the onset of damage to the optic nerve head and consequently do not prevent retinal ganglion cell death and optic nerve damage in glaucoma. Once initiated, the glaucomatous damage to the retinal ganglion cells occurs in a gradual yet progressive manner despite lowering the pressure. Moreover, these drugs only treat one form of glaucoma, primary open angle glaucoma (“POAG”) in which elevated intraocular pressure (“IOP”) may be a major symptom/cause for retinal ganglion cell death. However, in other glaucomas, like normal tension glaucoma (“NTG”), IOP is within the normal range of 15-20 mm Hg, yet the damage to the optic nerve and progression of retinal ganglion cell death is identical to that seen in POAG patients. In the glaucomatous retina, endothelins initiate a destructive cascade of downstream events (i.e. enhanced release of glutamate and nitric oxide) by either causing prolonged constriction of the retinal vasculature or through direct actions on neurotoxic substances, ultimately leading to retinal ganglion cell death. By preventing endothelin synthesis using ECE inhibitors or by blocking its actions with ET antagonists, one can directly target the source of damage and avert the potential cause of retinal ganglion cell death and optic nerve damage from occurring. The other advantage in using ECE inhibitors and/or ET antagonists is that they will prevent glaucomatous damage to the optic nerve irrespective of the etiology of the disease, as seen in different forms of glaucoma.

Presently, endothelin inhibitors in the form of endothelin receptor antagonists are widely used in the treatment of many cardiovascular diseases including congestive heart failure, ischemia and hypertension and in the treatment of subarachnoid hemorrhage in the brain. Presently, Bosentan (ETA/ETB receptor antagonist) is widely used as a drug to treat ET-mediated hypertension in clinical trials (Kiowski et al., 1995; Sutsch et al., 1997; Sutsch et al., 1998). Currently, there is no data available on the use of ECE inhibitors in treatment of ocular diseases (e.g. glaucoma). Thus, the invention disclosed herein will present compositions and methods for treating ocular diseases with ECE inhibitors and ET antagonists.

### SUMMARY

ECE inhibitors and/or ET antagonists administered topically, intracamerally (i.e. in front of the lens) or intravitreally (i.e. behind the lens in the vitreous cavity) can prevent optic nerve damage induced by ET in glaucoma. The use of ECE inhibitors or ET antagonists, which are currently tested for cardiovascular efficacy, have not been previously described for the treatment of glaucoma and represents a new use for these drugs. Inhibition of ECE will limit the production of endothelin in the eye whereas ET antagonists will block ET's actions. These drugs will reduce ET's ability to promote damage to the optic nerve and limit conditions that promote retinal ganglion cell death.

One aspect of the current invention involves a method for treating an ocular disease or damage thereof in an animal, comprising administering to the animal, a composition containing an effective amount of an endothelin-(“ET”) antagonist in a pharmaceutically acceptable vehicle. The ocular diseases or damage contemplated by the inventors are selected from the group consisting of ischemic retinopathies or optic neuropathies, commotio retinae, glaucoma, macular degeneration, retinitis pigmentosa, retinal detachment, retinal tears or holes, diabetic retinopathy, and iatrogenic retinopathy. The specific disease contemplated by the inventors is glaucoma.

An object of the current invention is to treat ocular disease or damage in an animal by administering one or more of ET-antagonists selected from a list of commercial products listed in Table 2. For example, a product available under the trade name of BOSENTAN, and has a formula of: 4-tert-butyl-N-[6-(2-hydroxy-ethoxy-5-2methoxy-phenoxy-[2,2′]-bipyrimidin-4-yl]-benzenesulfonamide monohydrate. A second aspect of a preferred ET antagonist is available under the trade name of PD142893 and has a formula of: N-Acetyl-b-phenyl D-Phy-Leu-Asp-Ile-Ile-Trp. A third aspect of a preferred ET antagonist available under the trade name of PD145065 and has a formula of: N-Acetyl-α-[10,11-Dihydro-5H-dibenzo[a,d]cycloheptadien-5-yl]-D-Gly-Leu-Asp-Ile-Ile-Trp. A fourth aspect of a preferred ET antagonist is available under the trade name of A192621 and has a formula of:

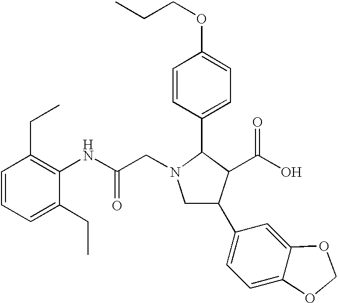

A fifth aspect of a preferred ET antagonist is available under the trade name of TBC10950 and has a formula of:

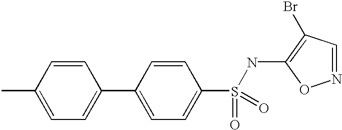

A sixth aspect of a preferred ET antagonist is available under the trade name of RO 46-8443 and has a formula of:

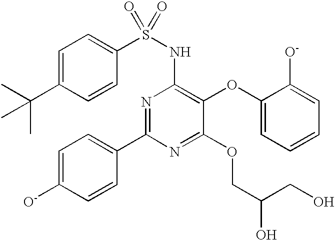

A seventh aspect of a preferred ET antagonist is available under the trade name of BQ788 and has a formula of: N—C is-2-6-Dimethyliperidinocarbonyl-L-gamma-methyleucyl-D-1-methylcarbonyl-tryptophanyl-D-Ile. A eighth aspect of a preferred ET antagonist available under the trade name of IRL1038 and has a formula of: Cys-Val-Tyr-Phe-Cys-His-Leu-Asp-Ile-Ile-Trp. A ninth aspect of a preferred ET antagonist is available under the trade name of TARASENTAN and has a formula of:

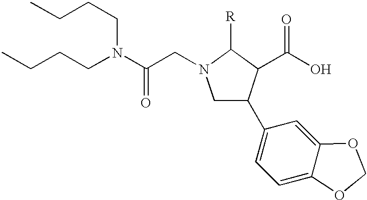

wherein R is C₂H₄—OCH₃. A ten aspect of a preferred ET antagonist available under the trade name of SITAXSENTAN and has a formula of:

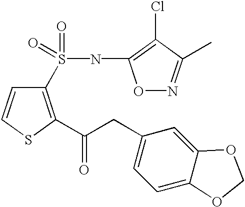

A tenth aspect of a preferred ET antagonist is available under the trade name of BQ-610 and has a formula of: (N,N-hexamethylene)-carbamoyl-Leu-D-Trp(CHO)-D-Trp. An eleventh aspect of a preferred ET antagonist is available under the trade name of BQ-123 and has a formula of c(D Trp-DAsp-Pro-DVal-Leu). A twelfth aspect of a preferred ET antagonist available under the trade name of BQ-788 and has a formula of N-cis-2,6-dimethylpiperidinocarbonyl-L-g-methyl-Leu-D-1-methoxycarbonyl-Trp-D-Nle and functions to inhibit an ET receptor.

Another object of the current invention involves a method for treating an ocular disease or damage thereof in an animal, comprising administering to the animal, a composition containing an effective amount of an endothelin-converting-enzyme (“ECE”) inhibitor in a pharmaceutically acceptable vehicle. The ocular disease or damage contemplated by the inventors are selected from the group consisting of ischemic retinopathies or optic neuropathies, commotio retinae, glaucoma, macular degeneration, retinitis pigmentosa, retinal detachment, retinal tears or holes, diabetic retinopathy, and iatrogenic retinopathy. The specific disease contemplated by the inventors is glaucoma.

The current invention contemplates a method for treating ocular disease or damage in an animal by administering one or more of ECE inhibitors selected from a list if commercial products listed in Table 1. For example, an effective ECE inhibitor comprises an aminophosphonate. A second example of an ECE inhibitor comprises an extract from a root of a Dalea filiciformis snader fabacae containing a compound with a general structure of:

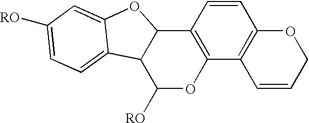

wherein, R is hydrogen, or COCH₃. A third example of an ECE inhibitor comprises a halistanol disulfate B, wherein the halistanol disulfate B comprises a sterol sulfate isolated from a pacharella sponge, wherein the sterol sulfate has a general structure of:

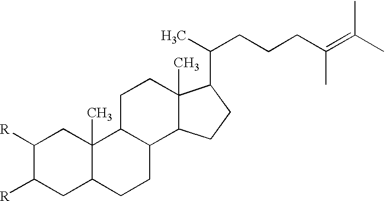

wherein, R is hydrogen, or SO₃H. A fourth aspect of an ECE inhibitor comprises a hydroxamic acid, wherein the hydroxamic acid is selected from a group consisting of: N-[2-[(hydroxyamino)carbonyl]-3-methylbutanoyl]-L-asparagine; N-[2-[(hydroxyamino)carbonyl]-3-methyl-1-oxobutyl]-β-aline; N-[2-[(hydroxyamino)carbonyl]-3-methylbutanoyl]-L-tryptophan; N-[2[(hydroxyamino)carbonyl]-3-methylpentanoyl]-L-tryptophan; and N-[2[(hydroxyamino)carbonyl]-3-methylbutanoyl]-L-glycine. A fifth aspect of an ECE comprises phosphoramidon is N-a-Rhamonopyranosyloxyhydroxyphosphinyl-Leu-Trp, wherein Leu is an abbreviation for the amino acid leucine, and wherein Trp is an abbreviation for the amino acid tryptophan. A sixth aspect of an ECE inhibitor comprises a benzo[α]naphtaeen chromophore, wherein the benzo[α]naphtaeen chromophore is 1,6,9,14-tertrahydoxy-3(2-hydroxypropyl)-7-methoxy-8,13-dioxo-5,6,8,13-tetrahydroenzo [α]naphthacene-2carboxylate-Na. A seventh aspect of an ECE inhibitor comprises a (S)-2biphenyl-4yl-1-(1H-tetrazol-5-yl)ethyl-amino-methyl compound. A seventh aspect of an ECE inhibitor comprises one or more compounds selected from the group consisting of: a daleformis; a halistanol disulfate B; a hydroxamic acid; a p-hydroxymercuribenzoate; and a phosphoramidon.

The methods contemplated by the inventors for administering the ET-antagonist or ECE-inhibitors to the animal are selected from a group consisting of systemic delivery, topical delivery, intraocular injection, intraocular perfusion, retrobulbar injection, intracameral delivery, intravitreal delivery. A method of treating an ocular disease in an animal, comprising: administering to the animal an effective amount of a mixture comprising a plurality of compounds, wherein the plurality of compounds comprise one or more ET antagonists and one or more ECE inhibitors has also been contemplated by the inventors.

### DESCRIPTION OF THE DRAWINGS

FIG. 1 shows endothelin's dual personality, in that the anterior chamber actions are beneficial, whereas events at the ONH are detrimental to retinal ganglion cells;

FIG. 2 shows the detection of endothelin converting enzyme-1 (ECE-1) in human non-pigmented ciliary epithelial (HNPE) cells and rat lung tissue;

FIG. 3 shows a bar graph depicting the time-dependent endothelin converting enzyme-1 (ECE-1) activity in HNPE cells and rat lung tissue as measured by a novel assay in which the enzyme converts ¹²⁵I Big ET-1 to ¹²⁵I ET-1;

FIG. 4 shows the effects of low and high doses of thiorphan, a dual inhibitor of NEP 24.11 and ECE-1, on ECE-1 activity in HNPE cells and rat lung tissue;

FIG. 5 shows effects of endothelin-1 (“ET-1”) on the mRNA expression of inducible nitric oxide synthase, NOS-2 in HNPE cells as determined by RT-PCR A representative gel figure is shown;

FIG. 6 shows the effect of endothelin-1 (“ET-1”) and PD-142893, an ET_{A/B} receptor antagonist, on nitric oxide (“NO”) release from human non-pigmented ciliary epithelial (HNPE) cells;

FIG. 7 shows endothelin-l's effects on the timed movements of radiolabeled materials through the most proximal 2-mm segment of the rat optic nerve;

FIG. 8 shows the effect of endothelin-1 on the distribution of radiolabeled material within the optic nerve for all times evaluated;

FIG. 9 shows the biphasic effect(s) of intravitreal ET-1's on anterograde axonal transport that were detected in all regions of the optic nerve;

FIG. 10 shows the effects of intravitreal ET-1 on anterograde axonal transport in rat optic nerve, for each time interval examined, expressed as corrected decays per minute (dpms);

FIG. 11 shows that no significant difference was observed between the effects of intravitreal ET-3 and intravitreal ET-1 on anterograde axonal transport at the 28 hour ISI;

FIG. 12 shows the effect of elevated intraocular pressure (“IOP”) on immunoreactive endothelin-1 (ir-ET-1) levels in the aqueous humor of rat model of glaucoma; and

FIG. 13 shows the intravitreal injection of endothelin-1 (2 mmoles) causes the mRNA expression of inducible nitric oxide synthase-2 (NOS-2) in the retina of Brown Norway rats either 4 hours or 24 hours post-injection as detected by RT-PCR.

### DETAILED DESCRIPTION OF THE PREFERRED EMBODIMENTS

Terms:

The term “Endothelin (“ET”)” as used herein refers to an encoded gene product in a family of three gene isopeptides ET-1, ET-2, and ET-3. ET peptides exhibit numerous biological activities both in vivo and in vitro acting via 2 membrane-bound receptors, viz. ETA and ETB. ET begins as an inactive 38-amino acid residue inactive peptide (“Big-ET”) that is cleaved by an Endothelin Converting Enzyme (“ECE”) to yield the active 21 amino acid residue ET-1.

The term “Endothelin Converting Enzyme (“ECE”)” as used herein refers is an enzyme that makes active ET.

The term “Human Ciliary Epithelium” as used herein refers to one type of cells responsible for the secretion of aqueous humor into the posterior chamber of the eye. The HNPE cells used in these experiments may represent a valuable resource for implementing the methods including dose ranges for treatment of ocular diseases outlined in the current invention. Ciliary epithelial tissues and retinal tissues from human donor eyes as well as from animals (e.g. rat model of glaucoma) will be used to complement the aforementioned.

The term “ET antagonist” as used herein refers to a compound that can inhibit ET function by contacting an ET receptor. Currently, many of these ET antagonists are used in the treatment of many cardiovascular diseases including congestive heart failure, ischemia and hypertension. Additionally, such treatments are used for subarachnoid hemorrhaging in the brain.

The term “animal” as used herein refers to any species of the animal kingdom. In preferred embodiments it refers more specifically to humans and all others known in the art.

The standard one- and three-letter abbreviations for amino acids used herein are as follows: Arginine, A, arg; Asparagine, R, asn; Aspartic acid, N, asp; Cysteine, C, cys; Glutamine, Q, gin; Glutamic acid, E, glu; Glycine, G, gly; Histidine, H, his; Isoleucine, I, ile; Leucine, L, leu; Lysine, K, lys; Methionine, M, met; Phenylalanine, F, phe; Proline, P, pro; Serine, S, ser; Threonine, T, thr; Tryptophan, W, trp; Tyrosine, Y, tyr; Valine, V, val.

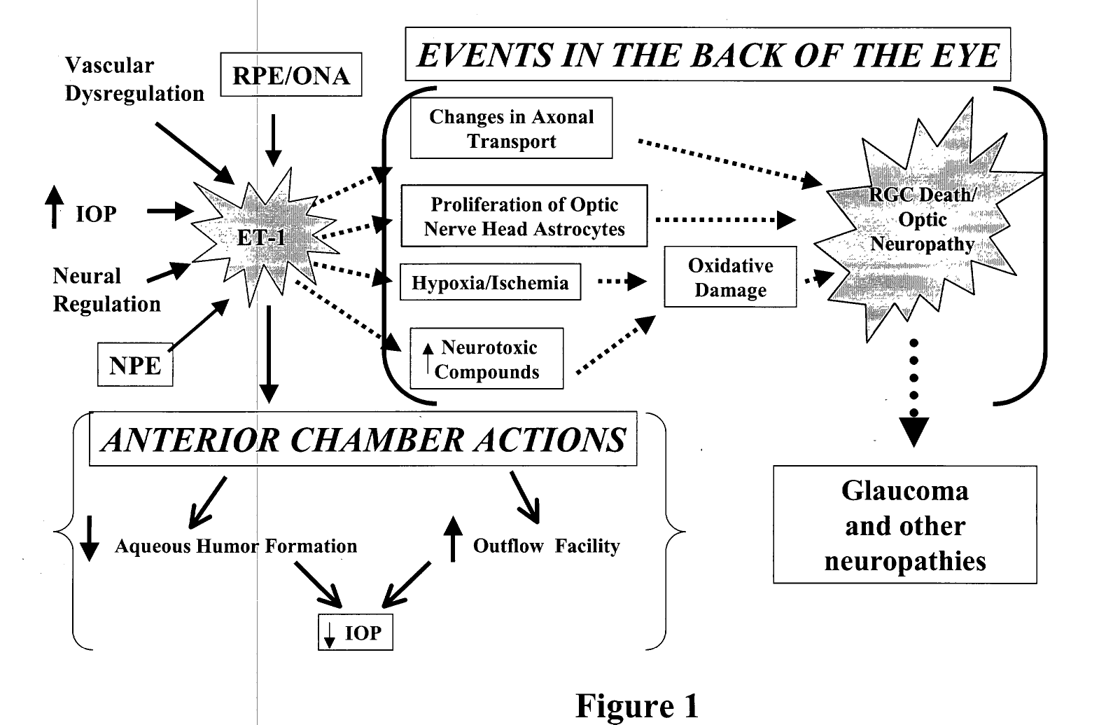

The vascular endothelium releases a variety of vasoactive substances, including an endothelium-derived vasoconstrictor peptide, endothelin (“ET”). ET is encoded by a family of three gene isopeptides ET-1, ET-2, and ET-3. ET acts via 2 known receptors, ETA and ETB, which are both abundantly expressed in the retina, optic nerve head, and aqueous humor inflow/outflow pathways in the eye. ET peptides exhibit numerous biological activities both in vivo and in vitro, and the effect of ET depends upon the amount of ET present. For example, our laboratory and others have shown that endothelin-1 in the anterior segment may have beneficial effects by contracting ciliary smooth muscle and increasing aqueous humor outflow while inhibiting Na⁺/K⁺ ATPase and decreasing aqueous humor formation. Such effects should decrease IOP; indeed, endothelin decreases IOP after intracameral or intravitreal administration (Sugiyama et al., 1995b; Taniguchi et al., 1994). Although not wanting to be bound by theory, our premise is that after increases in IOP in POAG, an increase in the synthesis and release of endothelin follows and in an attempt to reduce IOP by decreasing aqueous humor formation and enhancing outflow, as shown in FIG. 1. However, endothelin levels may be high in the back of the eye from retinal sources, and these increases in endothelin have detrimental and pathophysiological effects on the optic nerve, as has been seen with long-term endothelin administration and ischemia and during axonal transport after intravitreal administration (FIG. 1) (Ehrenreich et al., 1991; MacCumber and D'Anna, 1994). Furthermore, ET has been shown to increase inducible nitric oxide synthase-2 (“NOS2”) and NO concentrations in ocular tissues, which can have a further damaging effect on the optic nerve because of the production of peroxynitrites, as shown in FIG. 1. Three criteria suggest a pathophysiologic role for endothelin in glaucoma: 1) the increased circulating levels of endothelin that occur after decreased degradation, increased production, or delayed or no elimination; 2) the augmented responses to these increased endothelin concentrations by target cells expressing endothelin receptors and/or diminished counterbalancing mechanisms (e.g., reduction in vasodilator responses); and 3) the beneficial effects of anti-endothelin antibodies, selective endothelin receptor antagonists, or selective inhibitors of endothelin production in animal models or, ultimately, in managing disease pathology in humans. Regarding the role of endothelin in glaucoma, the first and second criteria have been shown in humans and in an animal model, and the third is the subject of this invention. The role of endothelin-1 and its elevated levels in normal-tension glaucoma, wherein the IOP is apparently normal, are conflicting. Although two reports show statistically significant elevation of plasma endothelin-1 levels in patients with normal tension glaucoma, another report shows only an insignificant trend, albeit differences in endothelin-1 levels when patients went from supine to upright positions (Cellini et al., 1997; Sugiyama et al., 1995a; Kaiser et al., 1995). FIG. 1 depicts the dual nature of endothelin, listing the pathways for beneficial effects in the anterior chamber and the detrimental effects on the optic nerve. It is noteworthy that carbachol, an analog of the neurotransmitter acetylcholine, increases the synthesis and release of this peptide from the ciliary process and retinal pigmented epithelial cells, which suggests a neuroregulatory control of endothelin synthesis and a possible link to the visual pathway.

ET provokes a strong and sustained vasoconstriction in vivo in rats and in isolated vascular smooth muscle preparations. ET also provokes the release of eicosanoids and endothelium-derived relaxing factor (“EDRF”) from perfused vascular beds. Intravenous administration of endothelin-1 and in vitro addition to vascular and other smooth muscle tissues produce long-lasting pressor effects and contraction, respectively. In isolated vascular strips ET-1 is slow acting, potent and persistent contractile agent. In vivo, a single dose of ET-1 can elevate blood pressure minutes. In the lung, endothelin-1 acts as a potent bronchoconstrictor. More recently patients with normotensive glaucoma and patients with primary open angle glaucoma (“POAG”) have elevated ET-1 concentrations in aqueous humor. Chronic elevation of ET levels in an area adjacent to the optic nerve has been shown to cause the vasoconstriction of the anterior optic nerve vasculature, which leads to optic nerve ischemia, and optic nerve damage.

ET begins as an inactive 38-residue inactive peptide (“Big-ET”) that is cleaved by an Endothelin Converting Enzyme (“ECE”) to yield the active 21 residue ET-1. By utilizing numerous ECE inhibitors that are commercially available, it is possible to prevent the production of active ET-1. Because the ciliary epithelium is responsible for the secretion of aqueous humor into the posterior chamber of the eye, human non-pigmented ciliary epithelial (“HNPE”) cells were used as a model to demonstrate how the use of ECE inhibitors can be utilized in ocular disease. Additionally, rat lung tissue compared with HNPE cells was used to show how ECE-inhibitors could inhibit the conversion of the inactive Big ET to the active form of ET (Table. 3).

Inhibition of ECE is a secondary method to prevent the inactive form of ET (Big ET) from becoming active. However, inhibition of ECE is only one method that will satisfactorily reduce the activity of ET. Another secondary method to alter ET activity is to inhibit the binding of the active ET from binding to the ET-receptor by contacting the receptor with an agonist. Additionally, endothelin inhibitors in the form of endothelin receptor antagonists are widely used in the treatment of many cardiovascular diseases including congestive heart failure, ischemia and hypertension and in the treatment of subarachnoid hemorrhage in the brain. Presently, Bosentan (ETA/ETB receptor antagonist) is widely used as a drug to treat ET-mediated hypertension in clinical trials (Kiowski et al., 1995; Sutsch et al., 1997; 1998). There are currently no data available on a method to use of ECE inhibitors in the treatment of ocular diseases.

The following examples are provided to further illustrate this invention and the manner in which it may be carried out. It will be understood, however, that the specific details given in the examples have been chosen for purposes of illustration only and not be construed as limiting the invention.

### EXAMPLE 1

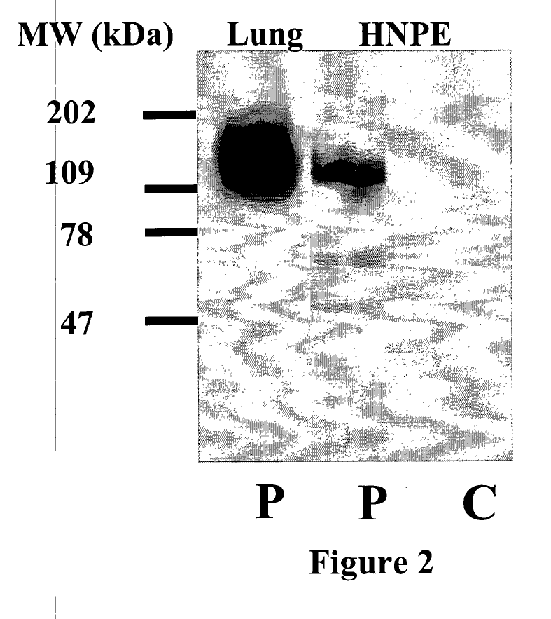

Detection of endothelin converting enzyme-1 (“ECE-1”) in the plasma membrane of human non-pigmented ciliary epithelial (HNPE) cells and rat lung tissue are shown in FIG. 2. Experimentally, approximately 75 μg total protein for HNPE and 8 μg for rat lung plasma membrane (“P”) fraction, and the cytosolic (“C”) protein fractions were separated by 7.5% SDS-PAGE under reducing conditions. The proteins were electrotransferred on to a nitrocellulose membrane polyclonal anti-rabbit against ECE-1 (1:500) and a secondary goat anti-rabbit antibody (1:20,000) was used to visualize the ECE-1 enzyme by a western blotting technique. Following a chemiluminescent treatment with alkaline phosphatase, the membranes were exposed to an X-ray film for 30 minutes to develop an image of the antibodies bound to the ECE-1 enzyme in the protein fractions. Dark bands represent the amount of ECE-1 that was detected. ECE-1 is shown as a 124-kDa protein only in the plasma membrane fraction (“P”) of both samples, but was not present in the cytosol fraction (“C”), rat lung cytosol lane is not shown. This experiment establishes that the majority of the ECE-1 enzyme is located in the plasma membrane of either HNPE cells or whole organ lung tissue. Therefore, only plasma membrane fractions will be assayed for the inhibition of ECE-1.

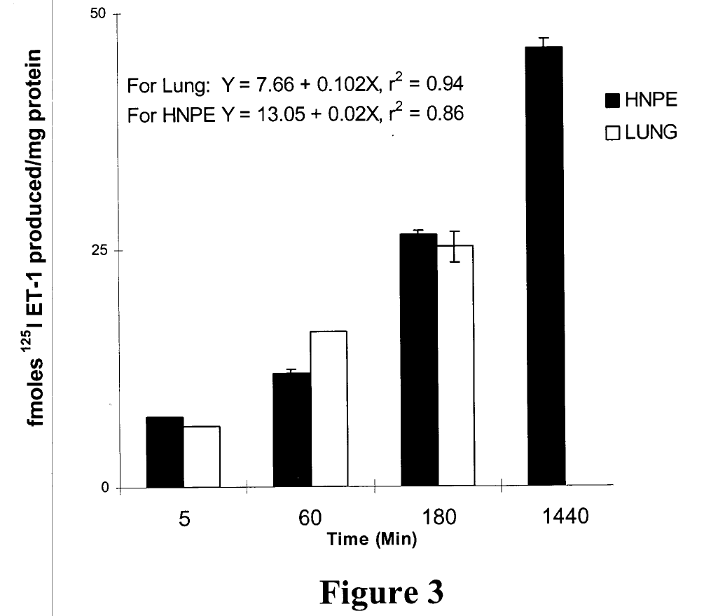

In replicate experiments, plasma membrane fractions (e.g. 20 μg total protein) of HNPE cells and rat lung tissue were incubated with 80 fmoles/mg protein of iodine 125 (“¹²⁵”) labeled 38 amino acid inactive endothelin peptide (“¹²⁵I Big ET-1”). Fractions were incubated at 37° C. and collected at various time periods ranging from 5 minutes to 24 hours. The endogenous endothelin converting enzyme (“ECE”) from the plasma membrane fraction converted the ¹²⁵I Big ET-1 to the shorter active ¹²⁵I ET-1 form which was detected by a novel assay. The results are shown in the bar graph of FIG. 3. A linear relationship in the conversion of ¹²⁵I Big ET-1 to ¹²⁵I ET-1 was observed with respect to time for both the HNPE cells and Lung tissue, as denoted by the linear regression equation in FIG. 3. Values represent the mean±SEM for data obtained from replicate samples. Statistical significance of mean [ET-1] among different time periods in HNPE cells as determined by one-way ANOVA and Student-Newman-Keuls multiple comparison test at p<0.05 (5 min n=3; 10 min n=6; 180 min n=9; 24 hrs n=4). Values that were statistically different are identified with an asterisk (“*”). Statistical significance of mean [ET−1] among different time periods in rat lung tissue as determined by one-way ANOVA and Student-Newman-Keuls multiple comparison test at p<0.05 (n=6 for 180 minutes; n=4 for other time periods) and denoted with a cross (“†”). It should be noted that the regression analysis (“r²”) and line equation (“y=m×+b”) for percent ECE-1 activity were calculated based on a line graph with equal scale on the X axis ranging from 0-1500 minutes.

Although an exhaustive list of ECE inhibitors exists, Table 1 illustrates a representation of selective commercially available ECE inhibitors and their respective IC₅₀ values. The ECE inhibitor CGS-26303 (Caner et al., 1996) with an IC₅₀ concentration in the range of 1-100 μM was used in an experiment similar to the one described above. Briefly, plasma membrane fractions (e.g. 20 μg total protein) of HNPE cells and rat lung tissue were incubated with 80 fmoles/mg protein of ¹²⁵I Big ET-1 for 180 minutes. The endogenous ECE from the plasma membrane fraction converted the ¹²⁵I Big ET-1 to the shorter active ¹²⁵I ET-1 form, which was detected as described previously. However, each of the samples was divided into two groups: a control; and the CGS 26303 treated group. Following the 180 minute incubation at 37° C., the samples treated with the ECE inhibitor CGS 26303 showed a diminished conversion of ¹²⁵I Big ET-1 to ¹²⁵I ET-1, as illustrated in Table 3. The HNPE plasma membrane fraction shows a 55% decrease in ¹²⁵I ET-1 production over the 180 minute time period. A 56% decrease in ¹²⁵I ET-1 was similarly observed in the rat lung plasma membrane fraction. Values in Table 3 represent the mean±SEM for data obtained from replicate experiments, as denoted in parentheses. The data were analyzed statistically by one-way ANOVA, and those values that show a significant difference (p<0.05 level) are identified with an asterisk (*).

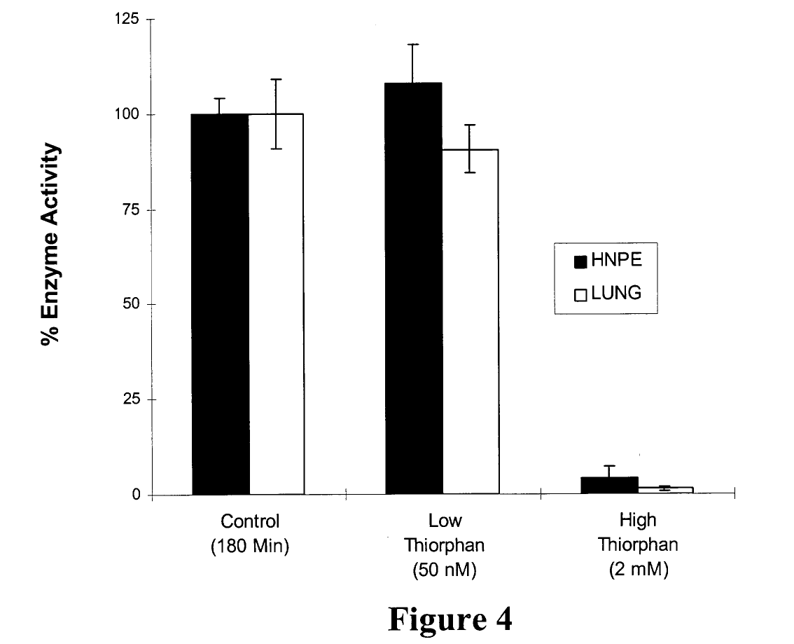

A similar experiment using low and high doses of thiorphan, a dual inhibitor of NEP 24.11 and ECE-1, on ECE-1 activity in HNPE cells and rat lung tissue was performed. Plasma membrane fractions of HNPE cells and rat lung tissue (20 μg total protein) were pre-incubated with thiorphan either at low (50 nM) or high (2 mM) doses for 30 minutes. Following the incubation period, ¹²⁵I Big ET-1 (80 fmole/mg protein; substrate) was added for 180 minutes at 37° C. while the control fraction did not contain the inhibitor. Results were recorded in FIG. 4 (HNPE: n=9 control; n=3 for both doses of thiorphan; Lung: n=3 for all three conditions). Percent enzyme activities in the treatments were compared to the control in which the amount of ¹²⁵I ET-1 produced was set at 100%. Statistical significance between control and high (2 mM) thiorphan treatment was determined by one-way ANOVA and those tissues that show a significant difference were further analyzed by Student-Newman-Keuls multiple comparison (p<0.05 level) and are identified with an asterisk (*). Because human ciliary epithelium is responsible for the secretion of aqueous humor into the posterior chamber of the eye, the HNPE cells in culture represents a valuable resource for implementing the methods including dose ranges for treatment of ocular diseases outlined in the current invention. Likewise, retinal pigmented epithelial cells, which also express ECE, are a potential source for ET in the retina and the optic nerve head. Retinal ET levels can be elevated due to increased ECE activity in glaucoma.

Exposure of cells to ET lead to the production of nitric oxide (“NO”). Nitric oxide is a gas molecule critical to numerous biological processes, including vasodilation, neurotransmission, as well as macrophage-mediated tumor, and microorganism killing processes. Nitric oxide is produced in an organism by enzymes. There are three different types of nitric oxide synthase (“NOS”): neuronal nitric oxide synthase (“NOSi”), inducible nitric oxide synthase (“NOS2”), and endothelium nitric oxide synthase (“NOS3”). Each have different tissue distributions and located on different human chromosomes.

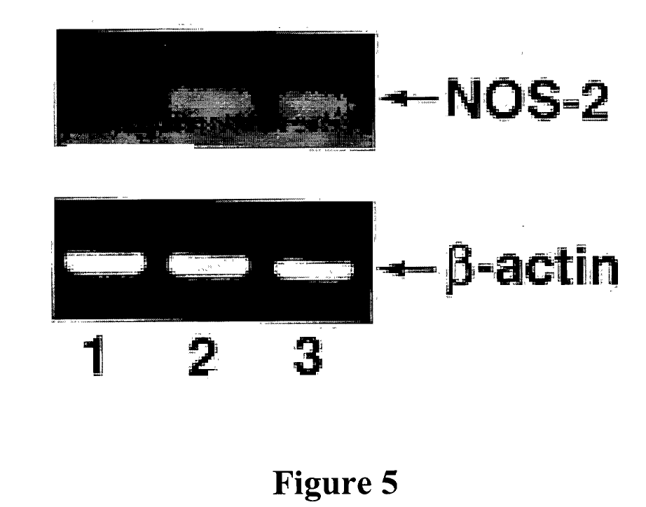

To demonstrate the effects of endothelin-i (“ET-1”) on the mRNA expression of inducible NOS2, HNPE cells exposed to cytokines and ET-1. Utilizing the powerful technique of RT-PCR on mRNA transcripts of NOS2, the amplified products were separated on an agarose gel and compared with untreated control RT_PCR products. As shown in FIG. 5, HNPE cells were treated for 24 hours with a) nothing (control lane 1), b) a cocktail of cytokines (IFN-γ+IL-1 +TNF-α) and lipopolysaccharide (lane 2) and c) 100 nM ET-1 (lane 3). RT-PCR products of the β-actin housekeeping gene were also amplified and visualized on an agarose gel as a mRNA isolation control. Qualitative examination of the NOS2 transcripts show that HNPE cells treated with ET or cytokines have elevated mRNA levels of NOS2 compared with non-treated controls. These results suggest that if ET-1 activity can be inhibited, NOS2 production can also be inhibited.

Although ECE inhibitors as listed in Table 1 may be used to prevent NOS2 production, other methods are available to reduce the activity of ET. For example, contacting an ET receptor with an antagonist will successfully inhibit the binding of the active ET to the ET-receptor. As shown in Table 2, many ET antagonists are commercially available. Currently, many of these ET antagonists are used in the treatment of many cardiovascular diseases including congestive heart failure, ischemia and hypertension. Additionally, such treatments are used for subarachnoid hemorrhaging in the brain.

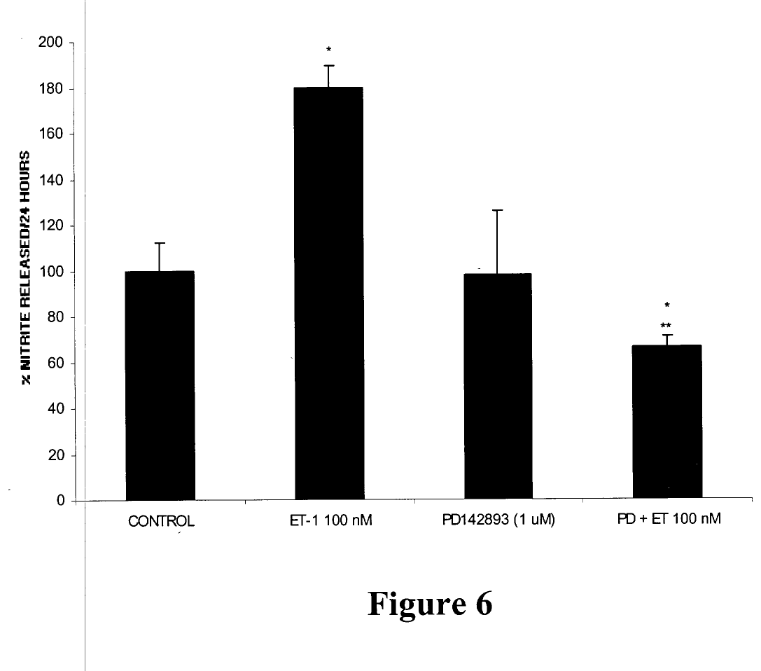

The effect of endothelin-1 (ET-1) and PD-142893, an ETA/B receptor antagonist, on nitric oxide (“NO”) release from human non-pigmented ciliary epithelial (HNPE) cells are illustrated in FIG. 6. The NO released into the culture media from HNPE cells grown in 24-well plates and treated with ET-1 (100 nM) for 24 hours was converted to nitrites. The total amount of nitrites was measured using the Griess colorimetric assay. HNPE cells were pre-treated with PD-142893 (1 μM) for 30 minutes and then 100 nM ET-1 was also added and incubated for 24 hours. The values in FIG. 6 represent the mean±SEM of a ratio between the nitrite produced under experimental conditions divided by the nitrite produced in control cells, which corresponded to 14.4±1.8 μM and controls were expressed as 100% (n=3). Statistical significance of the nitrite released from ET-1 (100 nM) treatment versus the control are denoted with an asterisk (“*”). Statistical significance of the nitrite released from PD-142893+ET-1 (100 nM) treatment versus that of ET-1 (100 nM) are denoted as a double asterisk (“**”) The statistical differenced were determined by one-way ANOVA and Student-Newman-Keuls multiple comparison test (p<0.05). Thus, addition of ET-1 increased the nitrite levels nearly 2-fold when compared to control cells or cells treated with PD-142893. In contrast, cells treated with both ET-1 and PD-142893 showed a decrease in nitrite production in the same 24-hour period.

### EXAMPLE 2

Newly synthesized proteins undergoing anterograde axonal transport in the optic nerve were pulse-labeled in either the presence or absence of endothelin as modified from previously published methods (van Biesen et al., 1995). Modifications to previously published methods were minimal and involved the replacement of a distilled water vehicle for resuspension of radiolabeled precursors with either HEPES buffered ET-1 or HEPES vehicle buffer alone. ³⁵S-Methionine (Easytag EXPRESS PROTEIN LABELING MIX, Dupont-NEN Life Sciences, Boston, Mass.) was lyophilized and resuspended in either vehicle alone (10 mM HEPES, pH 7.4, Sigma Chemical Co., St Louis, Mo.) or in vehicle containing 500 μM ET-1 (Bachem, Belmont, Calif.). Rats were anesthetized by Metofane inhalation, and 0.8 mCi (4 μl) of radiolabel in vehicle either plus or minus ET-1 (final dose 2 mmols), was injected into the vitreous of the left eye using a 30 gauge needle attached to a Hamilton syringe (microliter #710, 22s gauge, Hamilton Co., Reno, Nev.) by polyethylene tubing (PE-20, Clay Adams Brand, Becton Dickson and Co., Sparks, Md.) (van Biesen et al., 1995). In one experiment, ET-3 was substituted for ET-1, using the same methods (2 mmol dose, 28 hour ISI, N=7 for controls, N=7 for experimentals). During intravitreal injections, retinas were observed through the pupil with a Zeiss surgical microscope, model Stiffuss S. During introduction of the resuspended label into the vitreous, a transient blanching of the retina was observed for all animals, both controls and experimentals, which did not appear noticeably greater in the ET-1 treated animals, and began to recover immediately after the injection was complete. One minute after injection, all retinas appeared normal in color. (Based upon these initial observations, further observations of the retinas were not performed). Information on the dose-related effect(s) of intravitreal ET-1 in this species (i.e. rat) were unavailable, and physiological/pathological concentrations of endothelin in the optic nerve head's microenvironment are generally unknown. Therefore, dose selection was made on the basis of a small pilot study, using these methods and measuring the total pulse-labeled protein axonally transported into the rat optic nerve. (An N of 3 rats in every group was used only for the pilot study, 4 hour ISI, data not shown). The pilot study evaluated 0.3, 0.4, and 2 mmol doses of intravitreal ET-1 and showed a trend of increasingly enhanced axonal transport, compared to control, as the dose of ET-1 increased. However, significant effects on axonal transport (4 hour ISI) were only seen in the pilot study for the 2 mmol dose. The combination of a non-significant trend at lower doses with a large variance seen at the lowest significantly effective dose (2 mmols), was interpreted to mean that the 2 mmol dose was centrally located within the effective pharmacological dose range, for anterograde axonal transport in rat optic nerve, at the 4 hour ISI. Possible effect(s) on non-assayed ocular tissues were not considered in dose selection, as data on these were unavailable for either acute or chronic intravitreal ET-1 administration, in rats.

This technique was employed to determine if endothelin-1 also affected the axonal transport in the optic nerve of rats, which is severely compromised in glaucoma. Harvest and preparation of pulse-labeled optic nerves in animals, wherein animals were anesthetized with metofane at specified times after injection and sacrificed by decapitation. Injection-sacrifice intervals were selected based upon the published characterizations of anterograde axonal transport in rat optic nerve for specific marker proteins associated with specific classes of axonally transported materials (Table 4) (Elluru et al., 1995; Garner and Lasek, 1981; Garner and Lasek, 1982; Jahn et al., 1985). Seven vehicle-treated animals and seven endothelin-treated animals were sacrificed at each of the specified times (4, 24, 28, 32, 36 hours and 4, 21 days). Optic nerves were removed, flash frozen with crushed dry ice, and sectioned frozen. Nerves were sectioned to aid complete homogenization, and to provide additional data for future studies. The frozen sections (2 mm in length) were numbered as segments 1-4 (from proximal to distal), and glass-on glass homogenized in 100 μlof BUST sample buffer (2% β-mercaptoethanol, 8M urea, 1% SDS, 0.1M Tris, 0.02% phenol red, pH 7.4). A 25% aliquot of each homogenized segment was counted in a liquid scintillation counter, and counts were corrected for decay, quench, and counting efficiency.

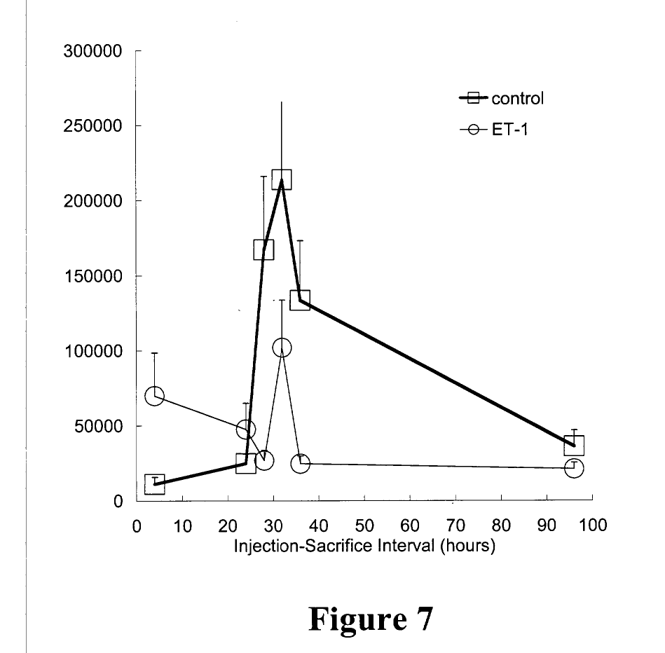

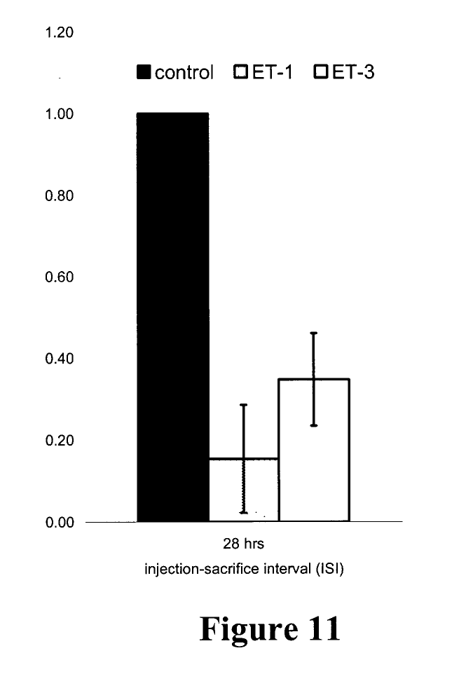

ET-1 caused alterations in all components of anterograde axonal transport. The effects of intravitreal endothelin-1 treatment were significant, biphasic, and prolonged (Table 4, p<0.05, and FIGS. 7-10, N=7 for controls, N=7 for experimentals, at each time point). The most profound effect of ET-1 was seen at 28 hours. At the 28 hour ISI, this effect was mimicked by the ET_{B}-receptor-selective agonist ET-3 (no significant difference between ET-1 and ET-3, p>0.999, ANOVA, N=7, FIG. 11), and suggests a receptor-mediated phenomenon, with similar effects for ET-1 and ET-3.

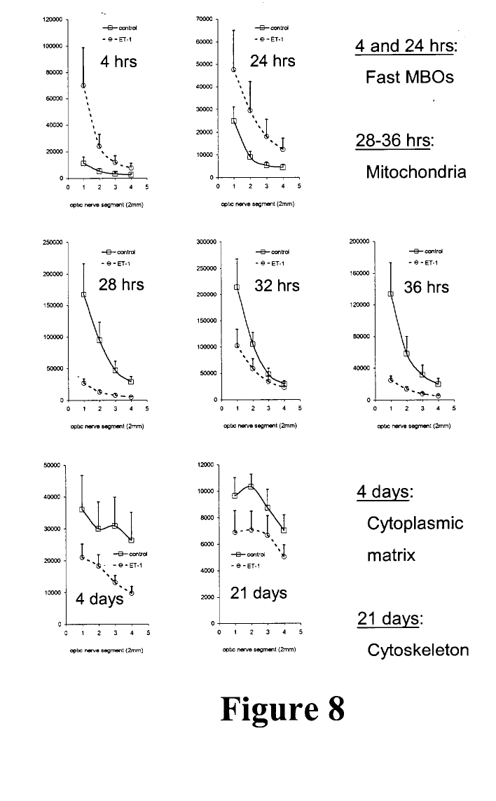

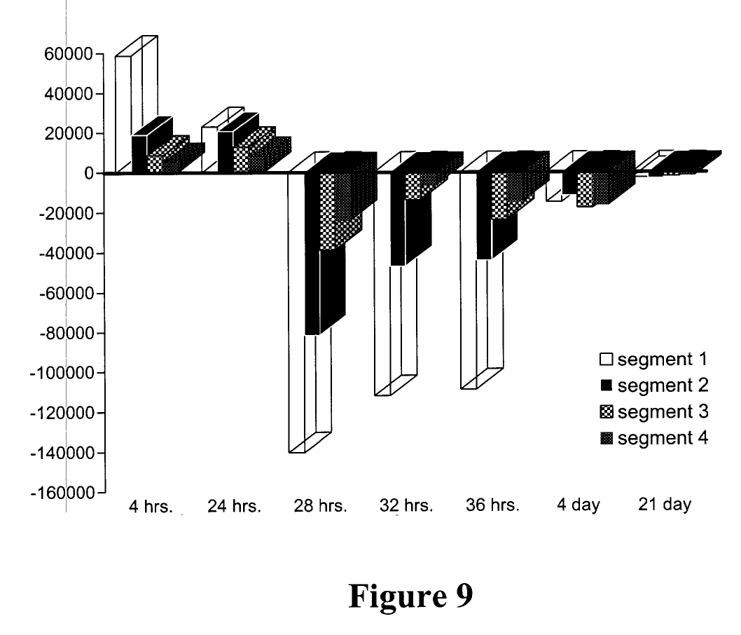

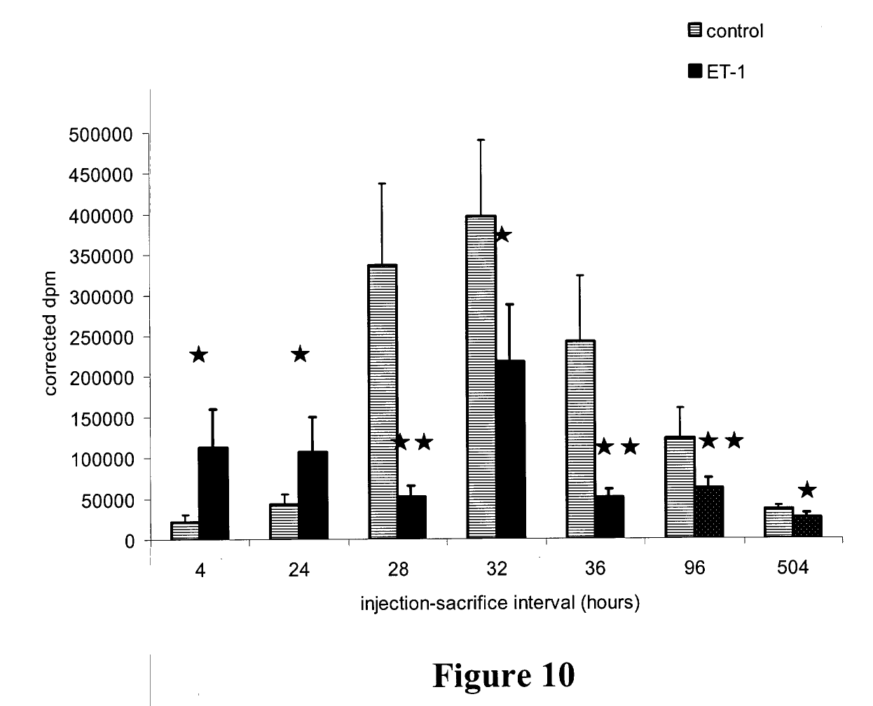

The direction and magnitude of ET-1's effects varied over time and with the cargo being transported (Table 4, FIGS. 9 and 10), suggesting a selective misregulation, as opposed to an indiscriminate inhibition, of anterograde axonal transport. Distributions of radiolabel within optic nerves were monitored (FIGS. 8 and 9) but all statistical comparisons (Table 4 and FIG. 10) were made on data for whole optic nerve (N=7 for each time point).

ET-1 moderately enhanced axonal transport of some small, fast tubulovesicles. There was a moderate, but significant enhancement of axonal transport into the optic nerve at those times normally associated with small, fast-moving tubulovesicles, but little or no mitochondrial marker proteins (4 and 24 hour ISIs, Table 4, FIGS. 7-10). The magnitude of ET-1's enhancement was greater for the 4 hour ISI than for the 24-hour ISI (FIG. 7). The 4 and 24 hour ISIs were selected for use in this study because they are normally associated with similar amounts of total anterogradely transported material, but the chemical compositions of transported material are different. Typically, a single form of the kinesin motor is associated with transport at the 4-hour ISI, while four different forms of the motor are associated with the 24-hour ISI. The possibility for differential regulation in the transport of various classes of tubulovesicles during this subcomponent motivated our use of the 4 and 24 hour ISIs. In this study, ET-1's effects on anterograde axonal transport at the 4 and 24 hour ISIs were not inconsistent with a hypothesized differential misregulation in the transport of various classes of small, fast tubulovesicles.

ET-1 severely decreased axonal transport in the mitochondrial subcomponent. Intravitreal ET-1's effects were most severe within the 28-36 hour window (Table 1, FIGS. 7, 9, and 10), causing a large reduction in transport at times when a large pulse (FIG. 7) of mitochondrial proteins normally moves through the rat optic nerve.

ET-1 moderately decreases axonal transport in the slow components. At times associated with both of the slow components of axonal transport, ET-1's effect(s) remained significant (Table 4), but were more moderate (FIGS. 8 and 10) than those seen during the mitochondrial subcomponent of fast transport (FIG. 8). This suggests a mechanism that is either somewhat component specific or partially reversible, and could result in less accumulation of cytoplasmic matrix and cytoskeletal proteins, than might be expected from a generalized loss of transport.

Biphasic effects of intravitreal ET-1. The effects of intravitreal endothelin-1 treatment upon anterograde axonal transport were biphasic (Table 4 and FIGS. 7, 9, and 10). The initial, rapid effect of ET-1 treatment was a significant enhancement of anterograde axonal transport into the optic nerve at 4 and 24 hours (FIGS. 7 and 10). The slower, but more prolonged effect of ET-1 was a significant reduction of anterograde axonal transport into the optic nerve at 28, 32, and 36 hours, 4 days, and 21 days (FIGS. 7 and 10).

Prolonged effects of ET-1. Intravitreal ET-1, administered as a single bolus, exerted significant effects (Table 4) upon anterograde axonal transport as early as 4 hours post-treatment (FIGS. 7 and 9) and as late as 21 days post-treatment (FIG. 10), inducing an extended period of aberrant axonal transport within the retinal ganglion cell axons of the optic nerve (FIG. 10). Chronic administration was not required to achieve this effect.

EXAMPLE 3

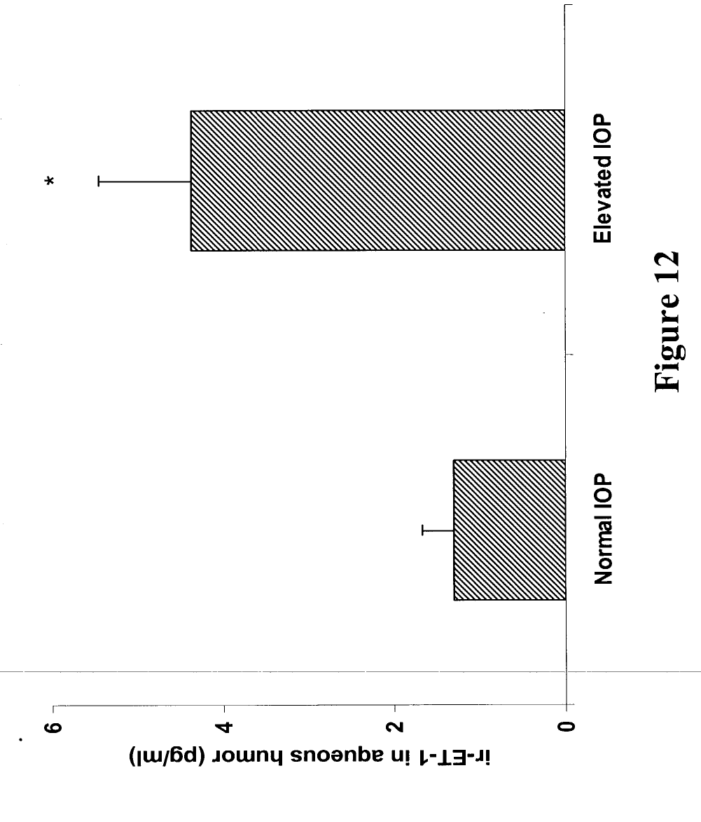

FIG. 12 shows the effect of elevated intraocular pressure (“IOP”) on immunoreactive endothelin-1 (ir-ET-1) levels in the aqueous humor of rat model of glaucoma. IOP was elevated in one eye of Brown Norway rats by injecting hypertonic saline into the episcleral veins using the method described by Morrison et al., (1997). The other eye served as an internal contralateral control. Another group of rats served as a control group. All rats were housed in vivarium under constant low light (<90lux) for up to 90 days. Following euthanasia, aqueous humor was collected, flash frozen, and stored at −80° C. until further use. A radioimmunoassay was used to determine the concentration of ET-1 in the aqueous humor samples. * Denotes statistical significance of ir-ET-1 in aqueous humor of elevated IOP rats versus a control group (also contralateral controls) at p<0.05 by Mann-Whitney U-test. (n=9 eyes for elevated IOP; n=12 eyes for normal IOP)

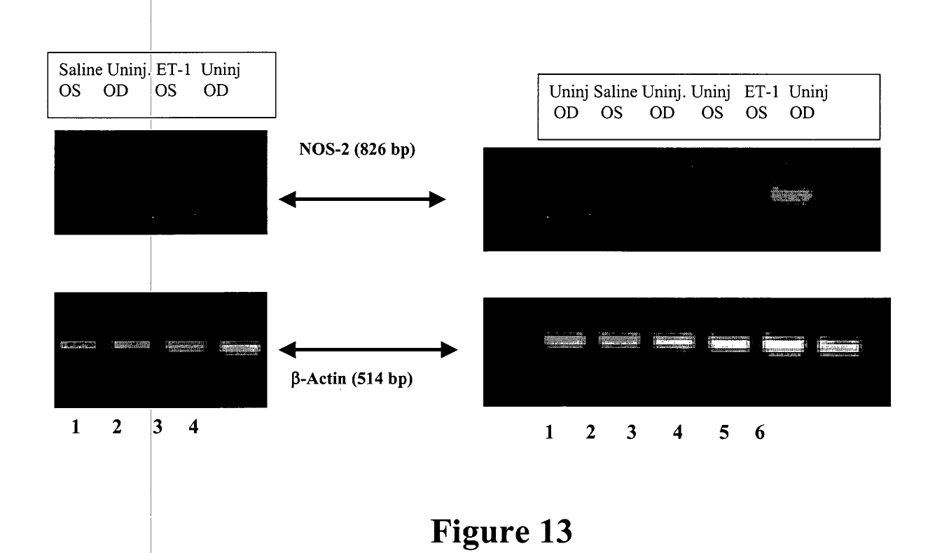

FIG. 13 shows the intravitreal injection of endothelin-1 (2 mmoles) causes the mRNA expression of inducible nitric oxide synthase-2 (NOS-2) in the retina of Brown Norway rats either 4 hours or 24 hours post-injection as detected by RT-PCR. Elevated NOS-2 expression and peroxynitrite labeling are observed in glaucomatous retina and optic nerve head and are considered to be neurotoxic, which promote retinal ganglion cell death. ET-1 was injected in the left eye (OS) of rats while the right eye served as an contralateral control. Saline was also injected in some rats. A group of rats also did not receive any injections. β-actin was used an internal control.

EXAMPLE 4

Based upon the physiological effects of endothelin (“ET”) and endothelin-converting enzyme (“ECE”) inhibitors, the present invention is directed toward compositions and method of applying ECE-1 inhibitors and ET antagonists as pharmaceutical agents to prevent optic nerve damage produced by endothelin in ocular disease (e.g. glaucoma). The compositions and methods of the present invention use agents which inhibit ECE, for preventing or protecting the retina and optic nerve head from diseases or damages caused by glaucoma, ischemia, trauma or edema.

ECE inhibitors may be administered systemically, topically, by intraocular injection, intraocular perfusion, periocular injection or retrobulbar injection. When ECE inhibitors are delivered by systemic administration, including oral administration, intramuscular injection, subcutaneous injection, intravenous injection, transdermal administration and transmucosal administration, the daily dosage of ECE inhibitors will range between 30 and 300 milligrams per kilogram body weight per day (mg/kg/day) and the effective doses will be between 70-280 mg/kg/day.

The exact dosage of one or more ECE inhibitor(s) to be administered to the patient will vary, but will be determined by clinicians skilled in the art. Various factors affecting the dosage amount include the actual disease to be treated, the severity of condition, the health of the patient, the availability of the active drug at the retina, potency and specific efficacy of the ECE inhibitor, and so on. The amount dosed, however, will be an “effective amount”. As used herein, the term “effective amount” is an amount, which inhibits ECE activity and subsequently active ET levels at a level effective for therapy.

The ECE inhibitors of the present invention may be contained in various types of ophthalmic compositions, in accordance with formulation techniques known to those skilled in the art. For example, the compounds may be included in solutions, suspensions and other dosage forms adapted for topical, intravitreal or intracameral use.

The ophthalmic compositions of the present invention will include one or more ECE inhibitor(s) of the present invention and a pharmaceutically acceptable vehicle. Aqueous solutions are generally preferred, based on ease of formulation and physiological compatibility. However, the ECE inhibitors of the present invention may also be readily incorporated into other types of compositions, such as suspensions, viscous or semi-viscous gels or other types of solid or semi-solid compositions. The ophthalmic compositions of the present invention may also include various other ingredients, such as buffers, preservatives, co-solvents and viscosity building agents. The active doses of ECE inhibitors that will be employed for topical application will range from 0.02%-5% (w/v).

An appropriate buffer system (e.g., hydrochloric acid/sodium hydroxide, sodium phosphate, sodium acetate or sodium borate) may be added to prevent pH drift under storage conditions.

Ophthalmic products are typically packaged in multidose form (2-15 ml volumes). Preservatives may be required to prevent microbial contamination during use. Suitable preservatives include: benzalkonium chloride, chlorohexidine, thimerosal, chlorobutanol, methyl paraben, propyl paraben, phenylethyl alcohol, edetate disodium, sorbic acid, polyquaternium-1, or other agents known to those skilled in the art. Some of these preservatives, however, may be unsuitable for particular applications (e.g., benzalkonium chloride may be unsuitable for intraocular injection or interference of preservatives with ECE inhibitors). Such preservatives are typically employed at a level of from is 0.001 to 1.0% weight/volume (“% w/v”).

At the present time there is evidence to suggest that topically administered drugs (used to lower IOP in glaucoma), may be able to gain access to the retina. However, there are no effective methods to directly target the back of the eye for chronic conditions via topical administration and it are presently contemplated that such methods will be developed. If topical administration of ECE inhibitors becomes feasible, the dosage generally will range between about 0.01% and 5% weight/volume (“w/v”), preferably between 0.25% and 1% (w/v). Solutions, suspensions, ointments, gels, jellies and other dosage forms adapted for topical administration are preferred. Similar dose ranges and effective doses as that for topical administration will be employed for the gel preparations. Additionally, ECE inhibitors may be delivered slowly, over time, to the afflicted tissue of the eye through the use of contact lenses. This regimen is generally performed by first soaking the lenses in a solution containing an ECE inhibitor and then applying the contact lenses to the eye for normal wear.

As used herein, the term “pharmaceutically acceptable carrier” refers to any formulation which is acceptable, i.e., safe and provides the appropriate delivery for the desired route of administration, of an effective amount of at least one ECE inhibitors of the present invention.

The compositions of the present invention are further illustrated in the following formulation examples, ECE inhibitors of the present invention are represented generically in the examples as “ECE Inhibitor”. However, the drugs listed in Tables 1 and Table 2 are representative agents in these classes. The invention includes any agent related in structure and pharmacology to these agents. These agents will be prepared for use in therapeutic effective concentrations for the treatment of ocular disease (e.g. glaucoma). According to the present invention, a therapeutically effective amount of ECE inhibitor is an amount sufficient to relieve or prevent optic nerve damage. Dosages can be readily determined by one of ordinary skill in the art and can be readily formulated into pharmaceutical dosing entities (i.e. pills, gels, drops, etc.).

### EXAMPLE 5

### A Topical Ophthalmic Composition Useful for Treating Ocular Neural Tissue:

| Component | % w/v |
|---|---|
| ECE Inhibitor | 0.25-1 |
| Dibasic Sodium Phosphate | 0.2 |
| HPMC | 0.5 |
| Polysorbate 80 | 0.05 |
| Benzalkonium Chloride | 0.01 |
| Sodium Chloride | 0.75 |
| Edetate Disodium | 0.01 |
| NaOH/HCl | q.s., pH 7.4 |
| Purified Water | q.s. 100% |

### EXAMPLE 6

A Sterile Intraocular Injection Solution Useful for Treating Ocular Neural Tissue:

| Component | % w/v |
|---|---|
| ECE Inhibitor | 0.25-1 |
| Cremophor EL. RTM. | 10 |
| Tromethamine | 0.12 |
| Mannitol | 4.6 |
| Disodiurn EDTA. | 0.1 |
| Hydrochloric acid or | q.s., pH to 7.4 |
| Water for injection | q.s. 100% |

### EXAMPLE 7

A tablet formulation suitable for oral administration, and useful for treating ocular neural tissue:

| Ingredient | Amount per tablet (mg) |
|---|---|
| ECE Inhibitor | 70-280 mg/kg/day |
| Cornstarch | 50 |
| Lactose | 145 |
| Magnesium stearate | 5 |

### EXAMPLE 8

An Systemic Injectable Solution Useful for Treating Ocular Neural Tissue:

| Ingredient | Amount |
|---|---|
| ECE Inhibitor | 70-280 mg/kg/day |
| 0.4 M KH₂PO₄ | 2.0 ml |
| 1 N KOH solution q.s. | to pH 7..0 |
| Water for injection | q.s. to 20 ml |

ET antagonist(s) may be administered systemically, topically, by intraocular injection, intraocular perfusion, periocular injection or retrobulbar injection. When ET antagonist(s) are delivered by systemic administration, including oral administration, intramuscular injection, subcutaneous injection, intravenous injection, transdermal administration and transmucosal administration, the daily dosage of ET antagonist(s) will range between about 20 and 200 milligrams per kilogram body weight per day (mg/kg/day), preferably between about 40 and 120 mg/kg/day.

The exact dosage of one or more ET antagonist(s) to be administered to the patient will vary, but will be determined by clinicians skilled in the art. Various factors affecting the dosage amount include the actual disease to be treated, the severity of condition, the health of the patient, the potency and specific efficacy of the ET antagonist(s), and so on. The amount dosed, however, will be an “effective amount.” As used herein, the term “effective amount” is an amount, which inhibits ET's activity at a level effective for therapy.

The ET antagonist(s) of the present invention may be contained in various types of ophthalmic compositions, in accordance with formulation techniques known to those skilled in the art. For example, the compounds may be included in solutions, suspensions and other dosage forms adapted-for topical, intravitreal or intracameral use.

The ophthalmic compositions of the present invention will include one or more ET antagonist(s) of the present invention and a pharmaceutically acceptable vehicle. Aqueous solutions are generally preferred, based on ease of formulation and physiological compatibility. However, the ET antagonist(s) of the present invention may also be readily incorporated into other types of compositions, such as suspensions, viscous or semi-viscous gels or other types of solid or semi-solid compositions. The ophthalmic compositions of the present invention may also include various other ingredients, such as buffers, preservatives, co-solvents and viscosity building agents.

An appropriate buffer system (e.g., hydrochloric acid/sodium hydroxide, sodium phosphate, sodium acetate or sodium borate) may be added to prevent pH drift under storage conditions.

Ophthalmic products are typically packaged in multidose form (2-15 ml volumes). Preservatives may be required to prevent microbial contamination during use. Suitable preservatives include: benzalkonium chloride, chlorohexidine, thimerosal, chlorobutanol, methyl paraben, propyl paraben, phenylethyl alcohol, edetate disodium, sorbic acid, polyquaternium-1, or other agents known to those skilled in the art. Some of these preservatives, however, may be unsuitable for particular applications, (e.g., benzalkonium chloride may be unsuitable for intraocular injection and/or potential interference with the drug). Such preservatives are typically employed at a level of from is 0.001 to 1.0% weight/volume (“% w/v”).

In topical administration of ET antagonist(s), the dosage generally will range between about 0.01% and 5% weight/volume (“w/v”), preferably between 0.25% and 1% (w/v). Solutions, suspensions, ointments, gels, jellies and other dosage forms adapted for topical administration are preferred. Additionally, ET antagonist(s) may be delivered slowly, over time, to the afflicted tissue of the eye through the use of contact lenses. This regimen is generally performed by first soaking the lenses in a solution containing an ET antagonist solution, and then applying the contact lenses to the eye for normal wear.

As used herein, the term “pharmaceutically acceptable carrier” refers to any formulation which is acceptable, i.e., safe and provides the appropriate delivery for the desired route of administration, of an effective amount of at least one ET antagonist(s) of the present invention.

The compositions of the present invention are further illustrated in the following formulation examples, ET antagonist(s) of the present invention are represented generically in the examples as “ET antagonist”. However, the drugs listed in Tables 1 and Table 2 are representative agents in these classes. The invention includes any agent related in structure and pharmacology to these agents. These agents will be prepared for use in therapeutic effective concentrations for the treatment of ocular disease (e.g. glaucoma). According to the present invention, a therapeutically effective amount ET antagonist is an amount sufficient to relieve or prevent optic nerve damage. Dosages can be readily determined by one of ordinary skill in the art and can be readily formulated into pharmaceutical dosing entities (i.e. pills, gels, drops, etc.).

### EXAMPLE 9

### A Topical Ophthalmic Composition Useful for Treating Ocular Neural Tissue:

| Component | %w/v |
|---|---|
| ET antagonist | 0.25-1 |
| Dibasic Sodium Phosphate | 0.2 |
| HPMC | 0.5 |
| Polysorbate 80 | 0.05 |
| Benzalkonium Chloride | 0.01 |
| Sodium Chloride | 0.75 |
| Edetate Disodium | 0.01 |
| NaOH/HCl | q.s., pH 7.4 |
| Purified Water | q.s. 100% |

### EXAMPLE 10

A Sterile Intraocular Injection Solution Useful for Treating Ocular Neural Tissue:

| Component | %w/v |
|---|---|
| ET antagonist | 0.25-1 |
| Cremophor EL. RTM. | 10 |
| Tromethamine | 0.12 |
| Mannitol | 4.6 |
| Disodium EDTA. | 0.1 |
| Hydrochloric acid or | q.s., pH to 7.4 |
| Water for injection | q.s. 100% |

### EXAMPLE 11

A tablet formulation suitable for oral administration, and useful for treating ocular neural tissue:

| Ingredient | Amount per tablet (mg) |
|---|---|
| ET antagonist | 40-120 mg/kg/day |
| Cornstarch | 50 |
| Lactose | 145 |
| Magnesium stearate | 5 |

### EXAMPLE 12

An Systemic Injectable Solution Useful for Treating Ocular Neural Tissue:

| Ingredient | Amount |
|---|---|
| ET antagonist | 40-120 mg/kg/day |
| 0.4 M KH₂PO₄ | 2.0 ml |
| 1 N KOH solution q.s. | to pH 7.0 |
| Water for injection | q.s. to 20 ml |

It should be appreciated by those of skill in the art that the techniques disclosed in the examples that follow represent techniques discovered by the inventor to function well in the practice of this invention, and thus can be considered to constitute preferred aspects for its practice. However, those of skill in the art should in light of the present disclosure, appreciate that many changes can be made in the specific embodiments which are disclosed and still obtain a like or similar result without departing from the spirit and scope of the invention.

For example, one of skill in the art may determine a therapeutically effective amount of ECE inhibitor or ET antagonist by measuring ECE activity for the former as shown in FIG. 3, and/or measuring ET levels in the aqueous humor as shown in FIG. 12, and in nitric oxide production for the latter as shown in FIG. 4.

While the compositions and methods of this invention have described in terms of preferred embodiments, it will be apparent to those of skill in the art that variations may be applied to the composition, methods and in the steps or in the sequence of steps of the method described herein without departing from the concept, spirit and scope of the invention. More specifically, it will be apparent that certain agents that are both chemically and physiologically related might be substituted for the agents described herein while the same or similar results would be achieved. All such similar substitutes and modifications to those skilled in the art are deemed to be within the spirit, scope and concept of the invention as defined by the appended claims.

### REFERENCES CITED

The following U.S. Patent documents and publications are incorporated by reference herein.

### U.S. PATENT DOCUMENTS

U.S. Pat. No. 6,342,610 issued on Jan. 29, 2002 with Chan et al. listed as inventors.

### OTHER REFERENCES

Ahn, K, A M Sisneros, S B Herman, S M Pan, D Hupe, C Lee, S Nikam, X M Cheng, A M Doherty, R L Schroeder, S J Haleen, S Kaw, N Emoto, M Yanagisawa, 1998, Novel selective quinazoline inhibitors of endothelin converting enzyme-1: Biochem. Biophys. Res. Commun., v. 243, p. 184-190.

Caner, H H, A L Kwan, A Arthur, A Y Jeng, R W Lappe, N F Kassell, K S Lee, 1996, Systemic administration of an inhibitor of endothelin-converting enzyme for attenuation of cerebral vasospasm following experimental subarachnoid hemorrhage: J. Neurosurg., v. 85, p. 917-922.

Cellini M, Possati G L, Profazio V, Sbrocca M, Caramazza N, Caramazza R. 1997. Color Doppler imaging and plasma levels of endothelin-1 in low-tension glaucoma. Acta Ophthalmol Scand Suppl. 224:11-3.

Cioffi, G A, S Orgul, E Onda, D R Bacon, E M Van Buskirk, 1995, An in vivo model of chronic optic nerve ischemia: the dose-dependent effects of endothelin-1 on the optic nerve microvasculature; Curr. Eye Res., v. 14,-p. 1147-1153.

Dreyer, E B, D Zurakowski, R A Schumer, S M Podos, S A Lipton, 1996, Elevated glutamate levels in the vitreous body of humans and monkeys with glaucoma: Arch. Ophthalmol., v. 114, p. 299-305.

Ehrenreich, H, R W Anderson, Y Ogino, P Rieckmann, T Costa, G P Wood, J E Coligan, J H Kehrl, A S Fauci, 1991, Selective autoregulation of endothelins in primary astrocyte cultures: endothelin receptor-mediated potentiation of endothelin-1 secretion: New Biol., v. 3, p. 135-141.

Elluru, R G, G S Bloom, S T Brady, 1995, Fast axonal transport of kinesin in the rat visual system: functionality of kinesin heavy chain isoforms: Mol. Biol. Cell, v. 6, p. 21-40.

Gamer, J A, R J Lasek, 1981, Clathrin is axonally transported as part of slow component b: the microfilament complex: J. Cell Biol., v. 88, p. 172-178.

Gamer, J A, R J Lasek, 1982, Cohesive axonal transport of the slow component b complex of polypeptides: J. Neurosci., v. 2, p. 1824-1835.

Hollander, H, F Makarov, Z Dreher, D van Driel, T L Chan-Ling, J Stone, 1991, Structure of the macroglia of the retina: sharing and division of labour between astrocytes and Muller cells: J. Comp Neurol., v. 313, p. 587-603.

Jahn, R, W Schiebler, C Ouimet, P Greengard, 1985, A 38,000-dalton membrane protein (p38) present in synaptic vesicles: Proc. Natl. Acad. Sci. U.S.A, v. 82, p. 4137-4141.

Kaiser H J, Flammer J, Wenk M, Luscher T. 1995. Endothelin-1 plasma levels in normal-tension glaucoma: abnormal response to postural changes. Graefes Arch Clin Exp Ophthalmol. 233(8):484-8.

Keller, P M, C P Lee, A E Fenwick, S T Atkinson, J D Elliott, W E DeWolf, Jr., 1996, Endothelin converting enzyme: substrate specificity and inhibition by novel analogs of phosphoramidon: Biochem. Biophys. Res. Commun., v. 223, p. 372-378.

Kiowski, W, G Sutsch, P Hunziker, P Muller, J Kim, E Oechslin, R Schmitt, R Jones, O Bertel, 1995, Evidence for endothelin-1-mediated vasoconstriction in severe chronic heart failure: Lancet, v. 346, p. 732-736.

MacCumber, M W, S A D'Anna, 1994, Endothelin receptor-binding subtypes in the human retina and choroid: Arch. Ophthalmol., v. 112, p. 1231-1235.

MacCumber, M W, H D Jampel, S H Snyder, 1991, Ocular effects of the endothelins. Abundant peptides in the eye: Arch. Ophthalmol., v. 109, p. 705-709.

Nathanson, J A, M McKee, 1995, Alterations of ocular nitric oxide synthase in human glaucoma: Invest Ophthalmol. Vis. Sci., v. 36, p. 1774-1784.

Neufeld, A H, 1999, Nitric oxide: a potential mediator of retinal ganglion cell damage in glaucoma: Surv. Ophthalmol., v. 43 Suppl 1, p. S129-S135.

Neufeld, A H, M R Hernandez, M Gonzalez, 1997, Nitric oxide synthase in the human glaucomatous optic nerve head: Arch. Ophthalmol., v. 115, p. 497-503.

Noske, W, J Hensen, M Wiederholt, 1997, Endothelin-like immunoreactivity in aqueous humor of patients with primary open-angle glaucoma and cataract: Graefes Arch. Clin. Exp. Ophthalmol., v. 235, p. 551-552.

Orgul, S, G A Cioffi, D J Wilson, D R Bacon, E M Van Buskirk, 1996, An endothelin-1 induced model of optic nerve ischemia in the rabbit: Invest Ophthalmol. Vis. Sci., v. 37, p. 1860-1869.

Sugiyama, T, S Moriya, H Oku, I Azuma, 1995a, Association of endothelin-1 with normal tension glaucoma: clinical and fundamental studies: Surv. Ophthalmol., v. 39 Suppl 1, p. S49-S56.

Sugiyama K, Haque MS, Okada K, Taniguchi T, Kitazawa Y. 1995b. Intraocular pressure response to intravitreal injection of endothelin-1 and the mediatory role of ETA receptor, ETB receptor, and cyclooxygenase products in rabbits. Curr Eye Res. 14(6):479-86.

Sutsch, G, O Bertel, W Kiowski, 1997, Acute and short-term effects of the nonpeptide endothelin-1 receptor antagonist bosentan in humans: Cardiovasc. Drugs Ther., v. 10, p. 717-725.

Sutsch, G, W Kiowski, X W Yan, P Hunziker, S Christen, W Strobel, J H Kim, P Rickenbacher, O Bertel, 1998, Short-term oral endothelin-receptor antagonist therapy in conventionally treated patients with symptomatic severe chronic heart failure: Circulation, v. 98, p. 2262-2268.

Taniguchi T, Okada K, Haque MS, Sugiyama K, Kitazawa Y. 1994. Effects of endothelin-1 on intraocular pressure and aqueous humor dynamics in the rabbit eye. Curr Eye Res. 13(6):461-4.

van Biesen T, Hawes B E, Luttrell D K, Krueger K M, Touhara K, Porfiri E, Sakaue M, Luttrell L M, Lefkowitz R J. 1995. Receptor-tyrosine-kinase- and G beta gamma-mediated MAP kinase activation by a common signaling pathway. Nature. 376(6543):781-4.

TABLE 1
Selective ECE inhibitors and their IC50 values.

| ECE Inhibitor (IC₅₀ in μM) | Reference |
|---|---|
| Aminophosphonates (0.3) | Fukami T et al. Bioorg. Med. Chem. Lett. |
| | 4:1257-1262, 1994. |
| CGS 25015 (18) | Trapani AJ et al. Biochem. Mol. Biol. Intl. |
| | 31:861-867, 1993. |
| CGS 26129 (60) | Trapani AJ et al. Biochem. Mol. Biol. Intl. |
| | 31:861-867, 1993. |
| CGS 26303 (1-100) | Caner HH et al. J. Neurosurg. |
| | 85:917-922, 1996. |
| Daleformis (9) | Patil AD et al. J. Natural Products. |
| | 60:306-308, 1997. |
| FR901533 (2-3) | Emoto N. and Yanagisawa M. J. Biol. |
| | Chem. 270:15262-15268, 1995 |
| Halistanol Disulfate B (2) | Patil AD et al. J. Natural Products. |
| | 59:606-608, 1996. |
| Hydroxamic acids | Bihovsky R et al. J. Med. Chem. |
| (low nM) | 38:2119-2129, 1995. |
| P-Hydroxymercuribenzoate | Deng Y et al. J. Biochem. |
| (1) | 111:346-351, 1992. |
| [Phe²²]-Big ET-1 [19-37] | Cliang A et al. J. Cardiovasc. Pharmacol. |
| (100) (analogue) | 26 (Suppl. 3):S72-S74, 1995. |
| * Phosphoramidon (0.14-1) | Keller PM et al. Biochem. Biophys. |
| | Res. Commun. 223:372-378, 1996. |
| PD 069185 (0.9) | Ahn K et al. Biochem. Biophys. Res. |
| | Commun. 243:184-190, 1998 |
| WS 79089 A (0.73) | Tsurumi Y et al. J. Antibiotics. |
| | 47:619-630, 1994. |
| WS 79089 B (0.14) | |
| WS 79089 C (3.4) | |
| WS 75624 A (low nM) | Tsurumi Y et al. J. Antibiotics |
| | 48:1066-1072, 1995. |
| WS 75624 B (low nM) | |
| Z-Phe-Phe-CHN₂ (1) | Deng Y et al. J. Biochem. |
| | 111:346-351, 1992. |

* There are several phosphoramidon-derivatives with substitutions to rhamnose, phosphoryl, leucine and tryptophan moieties of phosphoramidon that have various IC₅₀ values for ECE inhibition

TABLE 2
List of commercially available ET antagonists.

| | ET Re- | | ET | | ET Re- |
|---|---|---|---|---|---|
| Antagonist | ceptor | Antagonist | Receptor | Antagonist | ceptor |
| A-127722 | ET_{A} | Bosentan | ET_{A}/ET_{B} | A-192621 | ET_{B} |
| ABT-627 | ET_{A} | J-104132 | ET_{A}/ET_{B} | BQ-788 | ET_{B} |
| BQ-123 | ET_{A} | PD-142893 | ET_{A}/ET_{B} | IRL-1038 | ET_{B} |
| BE18257B | ET_{A} | RO-485695 | ET_{A}/ET_{B} | PD-145065 | ET_{B} |
| BQ-610 | ET_{A} | SB-209670 | ET_{A}/ET_{B} | | |
| FR-139317 | ET_{A} | TAK-044 | ET_{A}/ET_{B} | | |
| JKC-301 | ET_{A} | | | | |
| JKC-302 | ET_{A} | | | | |
| LU-135252 | ET_{A} | | | | |
| TBC-11251 | ET_{A} | | | | |

TABLE 3
Effect of CGS-26303, an ECE-1 inhibitor on ECE-1 activity in the
plasma membrane fractions of HNPE cells and rat lung tissue.

| Treatment (n) | fmoles ¹²⁵I ET-1 produced/mg protein/180 min |
|---|---|
| HNPE Cells | |
| Control (9) | 27 ± 1 |
| 100 μM CGS-26303 (5) | 12 ± 2* |
| Rat Lung Tissue | |
| Control (6) | 25 ± 1 |
| 100 μM CGS-26303 (4) | 11 ± 4* |

Each value represents mean ± S.D. of n samples.
*p < 0.05 vs. control

TABLE 4

| ISI | | | | effect of | |
|---|---|---|---|---|---|
| component | | | CARGO/subtype | ET-1 | p = |
| 4 | hr | fast | MBO/small tubulovesicles | increased | .010 |
| | | | | transport | |
| 24 | hr | fast | MBO/small tubulovesicles | increased | .020 |
| | | | | transport | |
| 28 | hr | fast | MBO/mitochondria | decreased | <.001 |
| | | | | transport | |
| 32 | hr | fast | MBO/mitochondria | decreased | .015 |
| | | | | transport | |
| 36 | hr | fast | MBO/mitochondria | decreased | <.001 |
| | | | | transport | |
| 4 | days | SCa | cytoplasmic matrix proteins | decreased | .001 |
| | | | | transport | |
| 21 | days | SCb | cytoskeletal proteins | decreased | .010 |
| | | | | transport | |

What is claimed is:

1. A method for treating an ocular disease or damage thereof in an animal, comprising administering to the animal, a composition containing an effective amount of an endothelin (“ET”) antagonist in a pharmaceutically acceptable vehicle, wherein the ocular disease or damage is selected from the group consisting of ischemic retinopathies or optic neuropathies, commotio retinae, glaucoma, macular degeneration, retinitis pigmentosa, retinal detachment, retinal tears or holes, diabetic retinopathy, and iatrogenic retinopathy.

2. The method of claim 1, wherein the ocular disease is glaucoma.

3. The method of claim 1, wherein the ET antagonist comprises 4-tert-butyl-N-[6-(2-hydroxy-ethoxy-5-2methoxy-phenoxy-[2,2′]-bipyrimidin-4-yl]-benzenesulfonamide monohydrate.

4. The method of claim 1, wherein the ET antagonist comprises N-Acetyl-β-Phenyl-D-Phe-Leu-Asp-Ile-Ile-Trp, wherein Phe is pheylalanine, Leu is leucine, Asp is aspartic acid, Ile is isoleucine, and Trp is tryptophan.

5. The method of claim 1, wherein the ET antagonist comprises N-Acetyl-α-[10,11-Dihydro-5H-dibenzo[a,d]cycloheptadien-5-yl]-D-Gly-Leu-Asp-Ile-Ile-Trp, wherein Gly is glycine, Leu is leucine, Asp is aspartic acid, Ile is isoleucine, and Trp is tryptophan.

6. The method of claim 1, wherein the ET antagonist comprises a compound with a formula of:

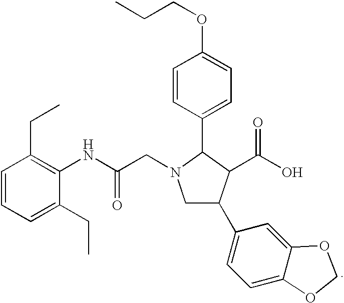

7. The method of claim 1, wherein the ET antagonist comprises a compound with a formula of:

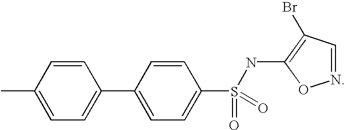

8. The method of claim 1, wherein the ET antagonist comprises a compound with a formula of:

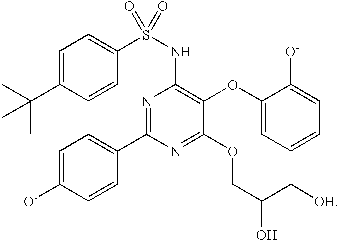

9. The method of claim 1, wherein the ET antagonist comprises N—C is-2-6-Dimethyliperidinocarbonyl-L-gamma-methyleucyl-D-1-methylcarbonyl-tryptophanyl-D-Ile, wherein Ile is isoleucine.

10. The method of claim 1, wherein the ET antagonist comprises Cys-Val-Tyr-Phe-Cys-His-Leu-Asp-Ile-Ile-Trp, wherein Cys is cystine, Val is valine, Tyr is tyrosine, Phe is Phenylalanine, His is histidine, Leu is leucine, Asp is asoartuc acid, Ile is isoleucine, and Trp is tryptophan.

11. The method of claim 1, wherein the ET antagonist comprises a compound with a formula of: 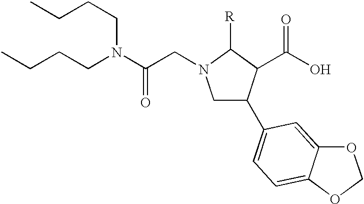
wherein R is C₂H₄—OCH₃.

12. The method of claim 1, wherein the ET antagonist comprises a compound with a formula of:

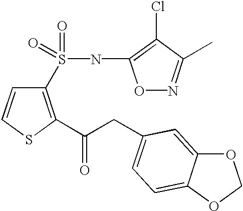

13. The method of claim 1, wherein the ET antagonist comprises (N,N-hexamethylene)-carbamoyl-Leu-D-Trp(CHO)-D-Trp, wherein Leu is leucine, Asp is aspartic acid, Ile is isoleucine, and Trp is tryptophan.

14. The method of claim 1, wherein the ET antagonist comprises c(D Trp-DAsp-Pro-DVal-Leu), wherein Asp is aspartic acid, Pro is proline, Val is valine, Leu is leucine, and Trp is tryptophan.

15. The method of claim 1, wherein the ET antagonist comprises N-cis-2,6-dimethylpiperidinocarbonyl-L-g-methyl-Leu-D-1-methoxycarbonyl-Trp-D-Nle and functions to inhibit an ET receptor, wherein Leu is leucine, Trp is tryptophan, and Nle is norleucine.

16. The method of claim 1, whereby administering the effective amount of one the ET antagonist is selected from a group consisting of systemic delivery, topical delivery, intraocular injection, intraocular perfusion, and retrobulbar injection.

17. The method of claim 1, whereby administering the effective amount of one the ET antagonist is selected from a group consisting of retrobulbar injection, intracameral delivery, intravitreal delivery.

18. A method for treating ocular disease or damage thereof in an animal, comprising administering to the animal, a composition containing an effective amount of an endothelin-converting-enzyme (“ECE”) inhibitor in a pharmaceutically acceptable vehicle, wherein the ocular disease or damage is selected from the group consisting of ischemic retinopathies or optic neuropathies, commotio retinae, glaucoma, macular degeneration, retinitis pigmentosa, retinal detachment, retinal tears or holes, diabetic retinopathy, and iatrogenic retinopathy.

19. The method of claim 18, wherein the ocular disease or damage is glaucoma.

20. The method of claim 18, wherein the ECE inhibitor comprises an aminophosphonate.

21. The method of claim 18, wherein the ECE inhibitor comprises a daleformis wherein the daleformis comprises an extract from a root of a Dalea filiciformis snader fabacae containing a compound with a general structure of: 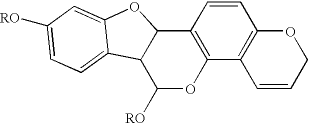
wherein, R is hydrogen, or COCH₃.

22. The method of claim 18, wherein the ECE inhibitor comprises a halistanol disulfate B, wherein the halistanol disulfate B comprises a sterol sulfate isolated from a pacharella sponge, wherein the sterol sulfate has a general structure of: 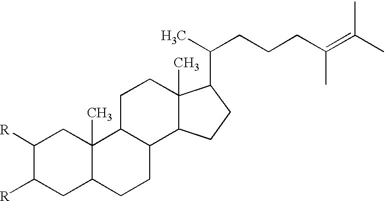
wherein, R is hydrogen, or SO₃H.

23. The method of claim 18, wherein the ECE inhibitor comprises a hydroxamic acid, wherein the hydroxamic acid is selected from a group consisting of:
(a) N-[2-[(hydroxyamino)carbonyl]-3-methylbutanoyl]-L-asparagine;
(b) N-[2-[(hydroxyamino)carbonyl]-3-methyl-1-oxobutyl]-β-aline;
(c) N-[2-[(hydroxyamino)carbonyl]-3-methylbutanoyl]-L-tryptophan;
(d) N-[2-[(hydroxyamino)carbonyl]-3-methylpentanoyl]-L-tryptophan; and
(e) N-[2-[(hydroxyamino)carbonyl]-3-methylbutanoyl]-L-glycine.

24. The method of claim 18, wherein the ECE inhibitor is a phosphoramidon and is N-a-Rhamonopyranosyloxyhydroxyphosphinyl-Leu-Trp, wherein Leu is leucine and Trp is tryptophan.

25. The method of claim 18, wherein the ECE inhibitor comprises a benzo[α]naphtaeen chromophore.

26. The method of claim 18, wherein the benzo[α]naphtaeen chromophore is 1,6,9,14-tertrahydoxy-3(2-hydroxypropyl)-7-methoxy-8,13-dioxo-5,6,8,13-tetrahydroenzo[α]naphthacene-2-carboxylate-Na.

27. The method of claim 18, wherein the ECE inhibitor comprises a (S)-2-biphenyl-4yl-1-(1H-tetrazol-5-yl)ethyl-amino-methyl compound.

28. The method of claim 18, wherein the composition comprises one or more compounds selected from the group consisting of:
(a) a daleformis;
(b) a halistanol disulfate B;
(c) a phosphonic acid;
(d) hydroxamic acid; and
(e) a phosphoramidon.

29. The method of claim 18, wherein the composition comprises p-hydroxymercuribenzoate.

30. The method of claim 18, whereby administering the effective amount of one the ECE inhibitor is selected from a group consisting of systemic delivery, topical delivery, intraocular injection, intraocular perfusion, and retrobulbar injection.

31. The method of claim 18, whereby administering the effective amount of one the ECE inhibitor is selected from a group consisting of retrobulbar injection, intracameral delivery, and intravitreal delivery.

32. A method of treating an ocular disease in an animal, comprising: administering to the animal an effective amount of a mixture comprising a plurality of compounds, wherein the plurality of compounds comprise one or more endothelin-(“ET”) antagonists and one or more endothelin-converting-enzyme (“ECE”) inhibitors.
